SEQUENCE LISTING (1 sequence)

SEQ ID NO: 1 - protein, 688 aa, Artificial Sequence
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGRIKEEDFIDRLLVANTHDHILCFSSRGRVYSMKVYQLPEATRGARGRPIVNLLPLEQDERITAILPVTEFEEGVKVFMATANGTVKKTVLTEFNRLRTAGKVAIKLVDGDELIGVDLTSGEDEVMLFSAEGKVVRFKESSVRAMGCNTTGVRGIRLGEGDKVVSLIVPRGDGAILTATQNGYGKRTAVAEYPTKSRATKGVISIKVTERNGLVVGAVQVDDCDQIMMITDAGTLVRTRVSEISIVGRNTQGVILIRTAEDENVVGLQRVAEPVDEEDLDTIDGSAAEGDDEIAPEVDVDDEPEEE

(19) United States
(12) Patent Application Publication
(10) Pub. No.: US 2016/0010144 A1
(43) Pub. Date: Jan. 14, 2016

(54) METHOD FOR SPECIFICALLY ISOLATING NUCLEIC ACIDS

(71) Applicant: FRAUNHOFER-GESELLSCHAFT ZUR FÖRDERUNG DER ANGEWANDTEN FORSCHUNG E.V., München (DE)

(72) Inventors: Dirk Kuhlmeier, Leipzig (DE); Natalia Sandetskaya, Leipzig (DE); Andreas Naumann, Leipzig (DE); Katharina Hennig, Krostitz (DE)

(21) Appl. No.: 14/438,418

(22) PCT Filed: Oct. 24, 2013
(86) PCT No.: PCT/EP2013/072331

§ 371 Date: Apr. 24, 2015

(30) Foreign Application Priority Data
Oct. 25, 2012 (DE) 10 2012 021 146.5

(51) Int. Cl. C12Q 1/68; C12N 9/90

(52) U.S. Cl. CPC C12Q 1/6806; C12N 9/90; C07K 2319/24; C12Y 599/01003

(57) ABSTRACT

The invention relates to an affinity material, comprising a carrier and an affinity protein bonded thereto, and a kit that comprises such an affinity material. The invention further relates to a method for separating and/or enriching prokaryotic DNA and the use of the affinity material to detect pathogenic bacteria or to detect a bacterial contamination.

The present invention relates to an affinity material for separating off and/or enriching prokaryotic DNA, which comprises a support and a protein bound thereto. The present invention additionally relates to a kit for enriching and/or separating prokaryotic DNA, which comprises such an affinity material, a method for separating off and/or enriching prokaryotic DNA, and also the use of the affinity material, the kit and/or the method in the field of molecular diagnostics, and environmental and food analysis.

The isolation of prokaryotic DNA is of great importance in many areas of analysis and diagnostics. For instance, the isolation, e.g. of pathogenic DNA for detection of a, in some circumstances, life-threatening infection, is an important challenge in molecular diagnostics. Pathogenic bacteria, even in low concentrations, can develop unwanted activity, but must, for reliable detection, frequently be cultivated in a complex manner and then characterized. In this case, for the patient affected, valuable time passes in which no effective therapy can be used. The invention is directed towards a method for the effective purification of bacterial nucleic acids from liquid sample media such as, e.g. blood, sputum or aqueous solutions, which is not only directed towards medical applications.

In the field of food analysis, also, methods are demanded which permit detection, which is as sensitive as possible, of a bacterial contamination. Here also, for reliable detection, complex intermediate steps for culturing the bacteria are frequently necessary, which leads to a long time period for such an analytical method and to high costs.

A particularly sensitive detection of bacteria succeeds by amplification of bacterial DNA by means of the polymerase chain reaction. However, in this case, it must be noted that the sensitivity and reliability of detection greatly depends on the ratio of the bacterial target nucleic acid to the background DNA.

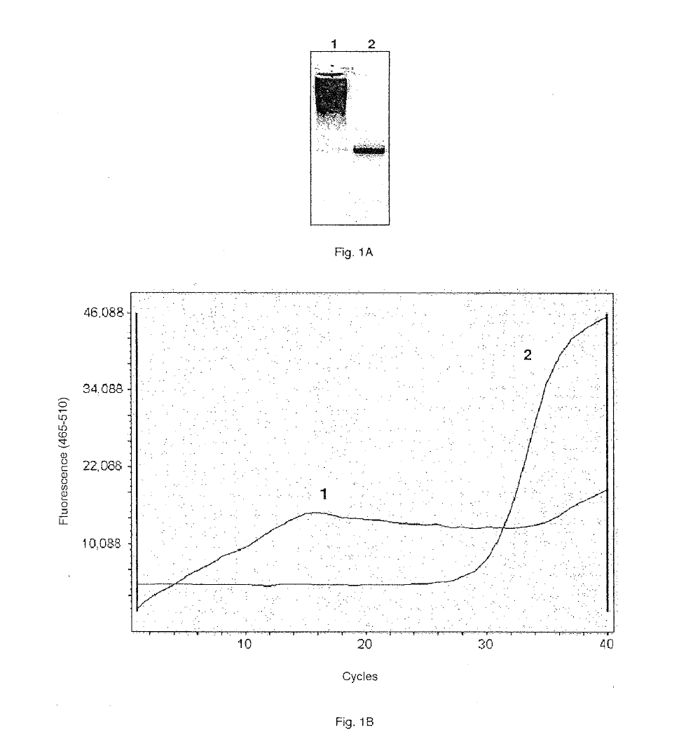

This is made clear by FIG. 1. FIG. 1 shows the detection of bacterial DNA in the presence (1) and absence (2) of human background nucleic acid. The latter total DNA was isolated from 1 ml of whole blood, spiked with 10⁶ cells of E. coli, whereas (2) was respectively generated only from an aqueous E. coli solution containing 10⁶ cells. FIG. 1 A shows analysis and detection by means of agarose gel electrophoresis, whereas FIG. 1 B shows a real-time PCR of the Lightcyclers© system (Roche). In both cases, the presence of the human nucleic acid generated a marked interference of the amplification reaction. Whereas in the gel image at least one unclear specific product band was visible for the bacterial DNA, via the fluorescence-optical method of real-time PCR, no clear analysis was obtained; the signal of curve 1 in FIG. 1 B was based on a non-specific product formation, which did not permit any conclusion about the bacterial DNA.

A high concentration of, e.g. human DNA (“background DNA”) leads to a markedly adverse effect on sensitivity. It is therefore necessary to isolate the bacterial DNA as completely as possible before carrying out the polymerase chain reaction, in order to separate off interfering contaminations or background DNA.

For isolating bacterial DNA from a sample, various methods are available in the prior art:

For the isolation of nucleic acids from samples, the affinity of DNA to silica materials is exploited. The silica matrix can be applied in this case to particles (e.g. magnetic particles) or as column material. The isolation methods customarily proceed according to the following plan:
a) cell lysis in the presence of chaotropic salts and proteinase (breakdown of protein) and subsequent centrifugation for separating off the cell residues
b) application to a column which contains silica material that interacts with the nucleic acid. The DNA is adsorbed on the surface, whereas other cell components are removed. The elution of the nucleic acid proceeds, after one or more washing steps, by adding water or low-salt buffer.

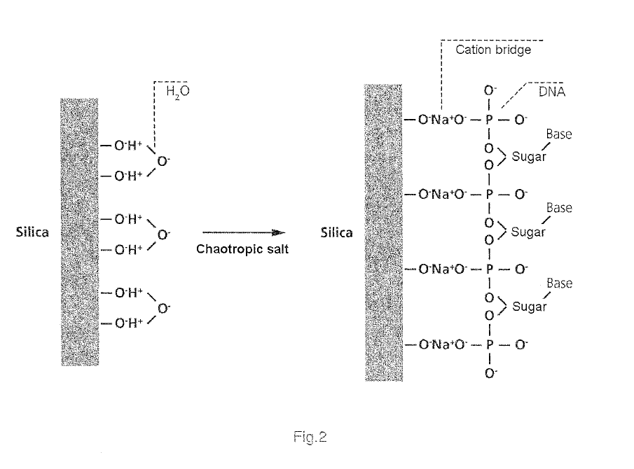

This method for the isolation of nucleic acids from samples by binding the DNA to silica materials is shown schematically in FIG. 2.

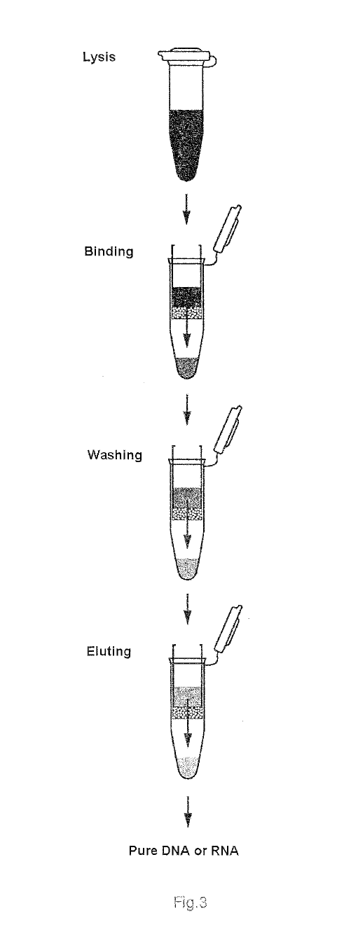

In practice, these methods require very many manual handling steps. In order to simplify the method sequence, for these methods, what are termed mini columns are frequently used. FIG. 3 shows schematically the sequence of such a method for isolating DNA by silica materials in a mini column.

After purifying the total nucleic acid with these silica materials, typically an amplification step follows, using which small concentrations of bacterial DNA, such as, e.g. in the detection of bacterial contaminants in human blood, can be detected. However, it has been found that the presence of numerous contaminations, in particular in the form of foreign DNA, markedly affects the limit of detection in this method (Handschur M, Karlic H, Hertel C, Pfeilstocker M, Haslberger A G. Preanalytic removal of human DNA eliminates false signals in general 16S rDNA PCR monitoring of bacterial pathogens in blood. Comp Immunol Microbiol Infect Dis. 2009 May; 32 (3): 207-19. Epub 2008 Feb. 7; Peters R P, van Agtmael M A, DNAer S A, Savelkoul P H, Vandenbroucke-Grauls C M. New developments in the diagnosis of bloodstream infections. Lancet Infect Dis. 2004 December; 4 (12); 751-60).

Thus this method for isolating nucleic acids from samples by affinity of the DNA to silica materials is associated with numerous disadvantages. For instance, the column materials used are generally associated with high costs. In addition, these methods require many manual steps, which occupy staff and are time consuming. Furthermore, these methods lead to purification of total DNA, which can contain an excess of irrelevant DNA. For instance, when the sample is obtained from a body fluid, the isolated bacterial DNA will generally be very highly contaminated with human DNA. The sensitivity of the detection of bacterial DNA by a subsequent polymerase chain reaction can be markedly decreased thereby.

In order to solve the problem of interfering foreign DNA, some methods are proposed in the prior art. In this case, firstly, methods are described in which a reduction of the foreign DNA is to proceed and secondly, methods are described in which an enrichment of the bacterial DNA is to take place.

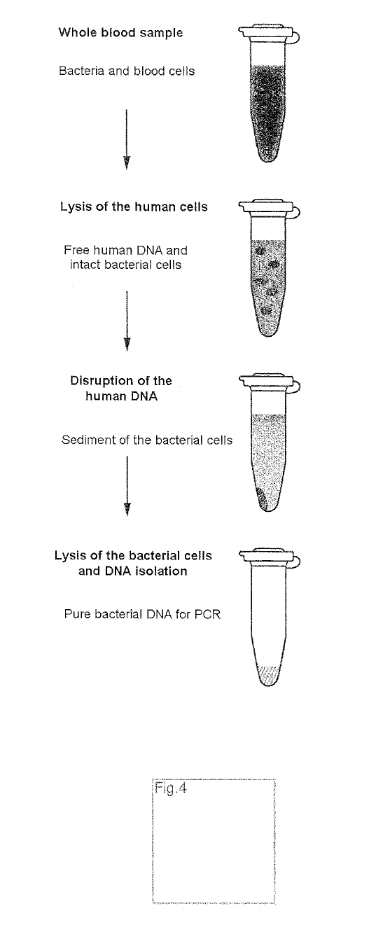

One example of a method for reducing the non-bacterial DNA includes the selective lysis of human cells with subsequent targeted breakdown of the human DNA. Such a method is shown schematically in FIG. 4.

FIG. 4 shows schematically the working sequence of a method for isolating bacterial nucleic acids from blood samples by targeted reduction of the human DNA. The suppliers market application kits for targeted depletion of human nucleic acid by lysis of the leukocytes. The liberated DNA is degraded in a targeted manner, with the bacterial cells remaining intact. Subsequently, the bacteria are sedimented with subsequent lysis of the bacterial cells for isolation of the bacterial DNA.

A disadvantage of this method is the complexity of the sequence, which makes the method time-consuming and labor-intensive. Furthermore, these methods are also associated with high costs.

In WO 2005/085440 A1, a method is described for separating off prokaryotic DNA by means of a protein that binds non-methylated CpG-motif-containing DNA specifically. In the method described in WO 2005/085440 A1, the property of prokaryotic DNA, of it differentiating itself from eukaryotic DNA by the occurrence of non-methylated CpG motifs, is utilized (cytidine-phosphate-guanosine dinucleotides) (Hartmann G et al., 2001, Deutsches Ärzteblatt Jg. 98/15: A981-A985 (2001).

In prokaryotic DNA, these motifs are non-methylated, whereas in eukaryotic cells they are for the most part methylated. For enriching bacterial DNA, in this method, a protein is used which specifically binds the non-methylated CpG motifs of a DNA. The protein hCpGBP (human CpG-binding protein) for this purpose is immobilized to Sepharose or similar matrices and incubated with the lysate from blood and potential pathogens.

However, this method is also associated with numerous disadvantages. For instance, this method only exhibits a weak enrichment of the bacterial DNA, since although with the hCpGBP protein, bacterial DNA is bound to a greater extent, significant binding of the more strongly methylated human DNA also still occurs. Therefore, the specificity of the separation method is rather low, which has an adverse effect on the reliability and sensitivity of a subsequent detection of the bacterial DNA. Furthermore, the method described in WO 2005/085440 A1 is rather costly.

Against the background of this prior art, the object of the present invention was to provide a material and a method which permits the separation off and/or enrichment of prokaryotic DNA from a sample. Compared with the previously known materials and methods, in this case a high degree of enrichment is intended to be achieved with as few as possible working steps and costs that are as low as possible.

The object mentioned is achieved according to the invention by an affinity material, comprising a support and a protein bound thereto, the protein comprising the DNA-binding subunit of a bacterial topoisomerase.

It has surprisingly been found that such an affinity material permits an extraordinarily specific separation off of bacterial DNA in a sample, especially even in the presence of large amounts of foreign DNA.

In an especially preferred embodiment, the protein comprises the subunit A of the topoisomerase II.

The invention utilizes the properties of the protein topoisomerase II (gyrase), which only occurs in prokaryotes.

The DNA-binding domain of gyrase A is used in order to isolate in a targeted manner bacterial nucleic acid from a mixture of highly complex DNA. Thereby, a general principle can be established in order to bind a broad spectrum of various DNAs reversibly and provided for further processes/analyses.

Topoisomerases have an important influence on the structure of the DNA macromolecule. Topoisomerase II is an enzyme from the group of topoisomerases which are ubiquitous and can loosen and dissolve helix coils of double-stranded DNA strands (source: Wikipedia). The bacterial gyrase is the only enzyme which, using ATP, can also induce negative supercoiling of a (ring-formed) DNA molecule. The protein consists of two subunits A and B which have different functionalities: GyrA has an active DNA binding site, whereas GyrB catalyzes the actual reaction of strand breaking and targeted relinking with consumption of ATP.

Especially preferably, in the context of the present invention the subunit gyrase A is used which can bind in a targeted manner to various bacterial sequences.

The use of this protein, after cell lysis and release of the total DNA, permits a selective binding of the bacterial DNA which can thereby be concentrated. As a result, especially human DNA, which is present naturally in many body samples, is bound only to a very slight extent.

In principle, the support used in the context of the present invention is not subject to any restriction. However, especially good results are achieved when the support is formed as magnetic particles, dextran, crosslinked polymers such as agarose and/or membrane. Especially preferably, in the context of the present invention, the support is magnetic particles.

Numerous possible methods are known for binding proteins to support materials. In the context of the present invention, the protein can be bound to the support both by covalent bonding and/or by noncovalent interaction, especially in the form of protein-protein interactions and/or protein-polysaccharide interactions.

It is especially advantageous when the protein used according to the invention, in addition to the DNA-binding domain of a bacterial topoisomerase, also contains a further protein component which can, for example, facilitate purification and/or improve the solubility of the protein.

In an especially preferred embodiment, the protein used according to the invention is therefore a fusion protein which comprises the DNA-binding subunit of a bacterial topoisomerase and an affinity tag.

Especially good results are achieved when the protein comprises a fusion protein from the subunit A of bacterial topoisomerase II and a maltose-binding protein.

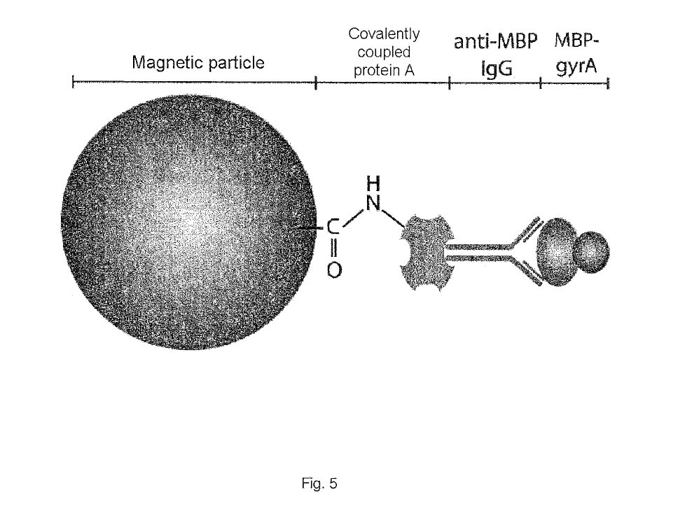

FIG. 5 shows a principle view of the immobilization strategy. Protein A binds covalently via formation of a peptide bond to the magnetic particles (MagPrep P-25 carboxy, Merck, Darmstadt). This protein fixes with high affinity an anti-MBP antibody which specifically immobilizes the fusion protein of gyrA and MBP via the Fv fragment of the antigen binding site (Fab region). The gyrA is thereby freely presented on the surface of the magnetic particle in order to trap bacterial nucleic acid in a targeted manner. The direct binding of the fusion protein to the surface of the magnetic particle is likewise possible in principle, in order to minimize the expenditure of the immobilization strategy.

It has been found that especially good results are achieved when the protein comprises a fusion protein from subunit A of bacterial topoisomerase II and a maltose-binding protein which has the amino acid sequence according to SEQ-ID No. 1:

```
                10 20 30 40
MKIEEGKLVI WINGDKGYNG LAEVGKKFEK DTGIKVTVEH
                50 60 70 80
PDKLEEKFPQ VAATGDGPDI IFWAHDRFGG YAQSGLLAEI
                90 100 110 120
TPDKAFQDKL YPFTWDAVRY NGKLIAYPIA VEALSLIYNK
              130 140 150 160
DLLPNPPKTW EEIPALDKEL KAKGKSALMF NLQEPYFTWP
              170 180 190 200
LIAADGGYAF KYENGKYDIK DVGVDNAGAK AGLTFLVDLI
              210 220 230 240
KNKHMNADTD YSIAEAAFNK GETAMTINGP WAWSNIDTSK
              250 260 270 280
VNYGVTVLPT FKGQPSKPFV GVLSAGINAA SPNKELAKEF
              290 300 310 320
LENYLLTDEG LEAVNKDKPL GAVALKSYEE ELAKDPRIAA
              330 340 350 360
TMENAQKGEI MPNIPQMSAF WYAVRTAVIN AASGRQTVDE
              370 380 390 400
ALKDAQTNSS SNNNNNNNNN NLGRIKEEDF IDRLLVANTH
              410 420 430 440
DHILCFSSRG RVYSMKVYQL PEATRGARGR PIVNLLPLEQ
              450 460 470 480
DERITAILPV TEFEEGVKVF MATANGTVKK TVLTEFNRLR
              490 500 510 520
TAGKVAIKLV DGDELIGVDL TSGEDEVMLF SAEGKVVRFK
              530 540 550 560
ESSVRAMGCN TTGVRGIRLG EGDKVVSLIV PRGDGAILTA
              570 580 590 600
TQNGYGKRTA VAEYPTKSRA TKGVISIKVT ERNGLVVGAV
              610 620 630 640
QVDDCDQIMM ITDAGTLVRT RVSEISIVGR NTQGVILIRT
              650 660 670 680
AEDENVVGLQ RVAEPVDEED LDTIDGSAAE GDDEIAPEVD
VDDEPEEE*
```

In a preferred embodiment of the affinity material according to the invention, the protein is formed as a fusion protein which has an amino acid sequence homology of ≧60%, especially preferably ≧80%, especially ≧90% with the amino acid sequence according to SEQ-ID No. 1.

Sequence homology in the context of the present invention is to be taken to mean especially the percentage of the identity of the amino acid sequences when two sequences are compared. In an especially preferred embodiment of the affinity material according to the invention, the protein is therefore formed as a fusion protein which has a sequence identity of ≧60%, especially preferably ≧80%, especially ≧90%, with the amino acid sequence according to SEQ-ID No. 1.

In order to produce the affinity material of the invention, the genetic information of the bacterial gyrA DNA-binding subunit was cloned and the protein expressed in E. coli. The N-terminal fused maltose-binding protein (MBP) was used for effective purification of the fusion protein. In modified electrophoretic mobility shift assays (EMSAs), the principle binding of DNA to the gyrA-MBP construct produced was able to be detected.

For detection of the specific binding of E. coli (Gram negative) and also Staphylococcus aureus (Gram positive) nucleic acid to the MBP-gyrA fusion protein, magnetic particles were functionalized with the trapping protein and incubated with DNA.

The DNA of both organisms was able to be purified in a targeted manner, even in the presence of human nucleic acid, and then amplified.

It has been found that the expression of the protein used according to the invention as fusion protein with the maltose-binding protein improves the solubility of the expressed protein and therefore facilitates the purification of the expressed protein.

The invention also relates to a kit which comprises the affinity material according to the invention.

In an especially preferred embodiment, the kit comprises means for amplifying the enriched/separated off prokaryotic DNA, especially in the form of primers for carrying out a polymerase chain reaction.

Furthermore, a kit according to the invention can also comprise further components, such as, for example, means for lysing the cells and for releasing the total DNA.

The present invention also relates to a method for separating off and/or enriching prokaryotic DNA, comprising the steps of:
a) contacting at least one prokaryotic DNA situated in solution with a binding agent that comprises the DNA-binding domain of a bacterial topoisomerase,
b) separating the prokaryotic DNA that is bound to the binding agent.

By such a method, the separation off and/or enrichment of prokaryotic DNA succeeds with high selectivity, even in the case of the presence of high amounts of foreign DNA in the sample.

In a preferred embodiment, the method according to the invention is formed in such a manner that the binding agent is present in the form of an affinity material comprising a support material and a protein bound thereto that comprises the DNA-binding subunit of a bacterial topoisomerase.

In a further preferred embodiment, the method according to the invention is formed in such a manner that the protein comprises the subunit A of bacterial topoisomerase II.

Especially good results are achieved when the method according to the invention is formed in such a manner that magnetic particles, Sepharose and/or dextran are used as support. It has been found that especially good results are achieved when magnetic particles are used as support.

The method according to the invention is preferably formed in such a manner that the protein is bound to the support by covalent bonding and/or by noncovalent interaction, especially in the form of protein-protein interactions and/or protein-polysaccharide interactions.

Preferably, the method according to the invention is formed in such a manner that the protein is a fusion protein which comprises a DNA-binding subunit of a bacterial topoisomerase and an affinity tag.

Especially good results are achieved when the protein comprises a fusion protein of the subunit A of the bacterial topoisomerase II and a maltose-binding protein.

It has been found that especially good results are achieved with the method according to the invention when the protein comprises a fusion protein from the subunit A of bacterial topoisomerase II and a maltose-binding protein that has the amino acid sequence according to SEQ-ID No. 1:

```
                10 20 30 40
MKIEEGKLVI WINGDKGYNG LAEVGKKFEK DTGIKVTVEH
                50 60 70 80
PDKLEEKFPQ VAATGDGPDI IFWAHDRFGG YAQSGLLAEI
                90 100 110 120
TPDKAFQDKL YPFTWDAVRY NGKLIAYPIA VEALSLIYNK
              130 140 150 160
DLLPNPPKTW EEIPALDKEL KAKGKSALMF NLQEPYFTWP
              170 180 190 200
LIAADGGYAF KYENGKYDIK DVGVDNAGAK AGLTFLVDLI
              210 220 230 240
KNKHMNADTD YSIAEAAFNK GETAMTINGP WAWSNIDTSK
              250 260 270 280
VNYGVTVLPT FKGQPSKPFV GVLSAGINAA SPNKELAKEF
              290 300 310 320
LENYLLTDEG LEAVNKDKPL GAVALKSYEE ELAKDPRIAA
              330 340 350 360
TMENAQKGEI MPNIPQMSAF WYAVRTAVIN AASGRQTVDE
              370 380 390 400
ALKDAQTNSS SNNNNNNNNN NLGRIKEEDF IDRLLVANTH
              410 420 430 440
DHILCFSSRG RVYSMKVYQL PEATRGARGR PIVNLLPLEQ
              450 460 470 480
DERITAILPV TEFEEGVKVF MATANGTVKK TVLTEFNRLR
              490 500 510 520
TAGKVAIKLV DGDELIGVDL TSGEDEVMLF SAEGKVVRFK
              530 540 550 560
ESSVRAMGCN TTGVRGIRLG EGDKVVSLIV PRGDGAILTA
              570 580 590 600
TQNGYGKRTA VAEYPTKSRA TKGVISIKVT ERNGLVVGAV
              610 620 630 640
QVDDCDQIMM ITDAGTLVRT RVSEISIVGR NTQGVILIRT
              650 660 670 680
AEDENVVGLQ RVAEPVDEED LDTIDGSAAE GDDEIAPEVD
VDDEPEEE*
```

In a preferred embodiment of the method according to the invention, the protein is formed as a fusion protein which has an amino acid sequence homology of ≧60%, especially preferably ≧80%, especially ≧90%, with the amino acid sequence according to SEQ-ID No. 1

Sequence homology, in the context of the present invention, is taken to mean especiallyl the percentage of the identity of the amino acid sequences when two sequences are compared. In an especially preferred embodiment of the method according to the invention, the protein is therefore formed as a fusion protein which has a sequence identity of ≧60%, especially preferably ≧80%, especially ≧90%, with the amino acid sequence according to SEQ-ID No. 1

The method according to the invention is suitable for analysis of a great multiplicity of samples which are to be examined for the presence of bacteria.

In a preferred embodiment, a body fluid is used as sample fluid, especially in the form of blood, serum, plasma, cell preparations from blood and/or sputum.

The method according to the invention, however, is in no way restricted to all applications for medical diagnostics. In an alternative embodiment, the method is used in such a manner that a fluid is used as sample fluid which is an environmental or food sample and/or a sample derived therefrom.

The method according to the invention is suitable, especially, for purifying bacterial DNA from a sample which is highly contaminated with foreign DNA of genomic origin. In an especially preferred embodiment, then, after the step of separation of the prokaryotic DNA bound to the binding material, a step c follows, in which the prokaryotic DNA is subjected to an amplification, especially by polymerase chain reaction. This sequence of method steps permits an extraordinarily sensitive and reliable detection even of small amounts of bacterial DNA in a sample.

The present invention also relates to the use of the affinity material or kit or method according to the invention for the detection of pathogenic bacteria for diagnostic purposes.

In an alternative embodiment, the present invention relates to the use of an affinity material or kit or method according to the invention for detection of a bacterial contamination in a food.

The material and method according to the invention have numerous surprising advantages in comparison with the prior art.

Especially, it has been found that the affinity material according to the invention has an extraordinarily high specificity for bacterial DNA in the presence of foreign DNA. Especially, the binding of contaminating human DNA to the affinity material according to the invention is very low, in such a manner that, in a simple manner, an extraordinarily high enrichment of the bacterial DNA is possible. The high specificity extends in this case not only to the bacterial DNA of Gram negative bacteria, but also of Gram positive bacteria. A further advantage is that there is a high flexibility with respect to the support material, as a result of which the material and method according to the invention can be used in a versatile and flexible manner. Furthermore, the affinity material according to the invention can be easily and inexpensively produced, and makes possible a method for separating off and/or enriching prokaryotic DNA which is executable rapidly and with few steps.

The invention described will be explained in more detail hereinafter with reference to examples:

### EXAMPLE 1

### Expression and Purification of the Fusion Protein gyrA-MBP

### Expression of the Fusion Protein GyrA-MBP

The C-terminal domain of the gyrase A subunit of E. coli (gyrA-CTD) was amplified by means of PCR using the primer pair gyrA BamH I fw/gyrA Stop Hind III rev. The vector pMK90 served as matrix for the PCR, which vector encodes the complete gyrase A subunit [Mizuuchi et al., 1984]. The amplicon was cloned for safeguarding into the pGEM-T vector and excised using BamH I and Hind III for subcloning. The target vector pMAL-C2× was likewise linearized using these restriction enzymes and then dephosphorylated. The gyrA-CTD fragment was finally ligated into the reader frame of the malE gene. Correct cloning was checked via sequencing.

### Purification of the Fusion Protein GyrA-MBP

At the start, 400 ml of LB/Amp/glucose (0.2%) medium were inoculated with 5 ml of a starter culture of the transformant. This culture was grown in the shaking incubator in a conical flask up to OD₆₀₀=0.6 (37° C.; 240 rpm). Protein expression was then induced by adding 450 μl of a 100 mM IPTG (isopropyl-β-D-thiogalacto-pyranoside) solution. Four hours post-induction, the culture was cooled to 4° C. and centrifuged down in the 50 ml reaction vessel (20 min; 4600 rpm; 4° C.). The pellet was then resuspended in 20 ml of column buffer A (100 mM HEPES (pH 7.5); 90 mM KCl; 1 mM MgCl₂, 1 mM TCEP (tris(2-carboxyethyl)phosphine), 1 mM PMSF (phenylmethylsulfonyl fluoride) and constantly cooled in the course of this on ice. The disruption was carried out using short ultrasound pulses (10 s) for 6 min. The cell remnants were separated off by centrifugation (30 min; 4600 rpm; 4° C.) and the supernatant transferred to a 50 ml reaction vessel.

In preparation for the purification, 1 ml of amylose matrix was charged onto a chromatography column and equilibrated with 16 ml of column buffer A. The supernatant was then added to the column and passed through the amylose matrix at a flow rate of 1 ml/min by gravity. Subsequently, the column was washed with column buffer A+0.2 M NaCl (4×3 ml).

The target protein was finally eluted from the matrix using 2×2 ml of column buffer A+10 mM maltose. The elution fractions, before further use, were dialyzed against 10 mM HEPES (pH 7.5) for 72 h at 4° C. They were stored at 4° C. The purification steps and purity were monitored by means of SDS-PAGE.

FIG. 6 shows a DNA-protein interaction analysis, with an EMSA being carried out (5% polyacrylamide gel, stained with ethidium bromide). The proteins gyrA-MBP and MBP were examined with specific oligonucleotides (56 bp, 0.25 pmol). The amounts of substance of the protein used respectively are stated: A-2.5 pmol, B-10 pmol, C-25 pmol.

As may clearly be seen in FIG. 6, strong binding of the fusion protein GyrA-MBP to the specific oligonucleotides used occurs.

The protein sequence of the construct is as follows:

MBP—gyrA (688 AA; M_{w}=75.15 kDa); theoretical pI 4.87)

```
                10 20 30 40
MKIEEGKLVI WINGDKGYNG LAEVGKKFEK DTGIKVTVEH
                50 60 70 80
PDKLEEKFPQ VAATGDGPDI IFWAHDRFGG YAQSGLLAEI
                90 100 110 120
TPDKAFQDKL YPFTWDAVRY NGKLIAYPIA VEALSLIYNK
              130 140 150 160
DLLPNPPKTW EEIPALDKEL KAKGKSALMF NLQEPYFTWP
              170 180 190 200
LIAADGGYAF KYENGKYDIK DVGVDNAGAK AGLTFLVDLI
              210 220 230 240
KNKHMNADTD YSIAEAAFNK GETAMTINGP WAWSNIDTSK
              250 260 270 280
VNYGVTVLPT FKGQPSKPFV GVLSAGINAA SPNKELAKEF
              290 300 310 320
LENYLLTDEG LEAVNKDKPL GAVALKSYEE ELAKDPRIAA
              330 340 350 360
TMENAQKGEI MPNIPQMSAF WYAVRTAVIN AASGRQTVDE
              370 380 390 400
ALKDAQTNSS SNNNNNNNNN NLGRIKEEDF IDRLLVANTH
              410 420 430 440
DHILCFSSRG RVYSMKVYQL PEATRGARGR PIVNLLPLEQ
              450 460 470 480
DERITAILPV TEFEEGVKVF MATANGTVKK TVLTEFNRLR
              490 500 510 520
TAGKVAIKLV DGDELIGVDL TSGEDEVMLF SAEGKVVRFK
              530 540 550 560
ESSVRAMGCN TTGVRGIRLG EGDKVVSLIV PRGDGAILTA
              570 580 590 600
TQNGYGKRTA VAEYPTKSRA TKGVISIKVT ERNGLVVGAV
              610 620 630 640
QVDDCDQIMM ITDAGTLVRT RVSEISIVGR NTQGVILIRT
              650 660 670 680
AEDENVVGLQ RVAEPVDEED LDTIDGSAAE GDDEIAPEVD
VDDEPEEE*
```

### EXAMPLE 2

### Production of GyrA-MBP Functionalized Magnetic Particles

650 μg protein A—magnetic particles (e.g. MyOne©, 1.05 μm, Invitrogen) were separated magnetically and the buffer supernatant discarded. The particles were coated with a monoclonal anti-MBP antibody (e.g. New England Biolabs), by resuspending them in 200 μl of solution ((1:46) in PBS/Tween 20) and incubated for 10 min at room temperature. Subsequently, the particles were washed once each with PBS/Tween 20 and gyrA-binding buffer (10 mM HEPES (pH 7.5); 50 mM KCl, 4 mM MgCl₂; 1 mM DTT) and after completion resuspended in 40 μl of GyrA—binding buffer.

For the principle structure of the construct, reference is again made to FIG. 5.

### EXAMPLE 3

### Isolation of S. aureus and E. coli DNA from Buffer Samples Using the Magnetic Particles Functionalized with GyrA-MBP

200 μl of GBBT buffer (10 mM HEPES, 50 mM KCl, 4 mM MgCl₂, 1 mM DTT, 0.05% Tween 20, pH 7.5) were spiked with differing concentrations (0.00005 ng to 0.5 ng) of bacterial genomic DNA. By way of a model, 20 μg of human DNA were added, in order to simulate DNA samples which had been extracted from infected biological material. 200 μg of gyrA-coated beads were added to the sample and samples were incubated for 30 min at 37° C. with constant shaking. The beads were washed three times with 10 mM tris-HCl pH 8.5 and resuspended in 9 μl of water. For DNA isolation, the beads were heated for 10 min at 95° C.

FIG. 7 shows an agarose gel electrophoresis (1.5% gel, stained with ethidium bromide) of the PCR product after carrying out the binding assay without (control) and with prokaryotic (E. coli; S. aureus) and eukaryotic (human) genomic DNA (gDNA).

M: Marker (GeneRuler Low Range DNA Ladder, Fermentas); A: binding assay carried out using the respective gDNA; R: reference, PCR of the respective gDNA as positive control; +: components added; —: component not present; the DNA fragments were amplified using the respective primers of the gDNA to be detected: E. coli 16S rRNA (353 bp; Gram neg Primer (E. coli), DG74), S. aureus 16S rRNA (215 bp; 16S_Stau_{—}95-110_F, 16S_Stau_{—}284-300_R), hGAPDH (240 bp; hGAPDH_neu_fw, hGAPDH_neu_rev).

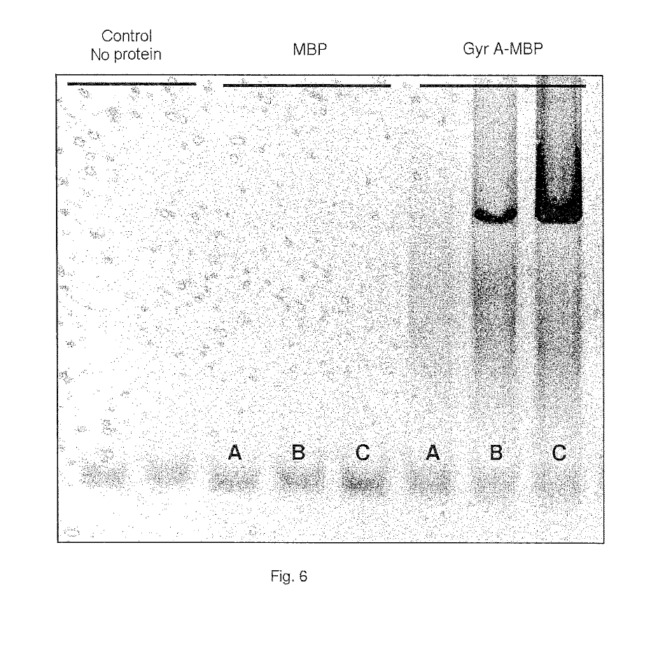

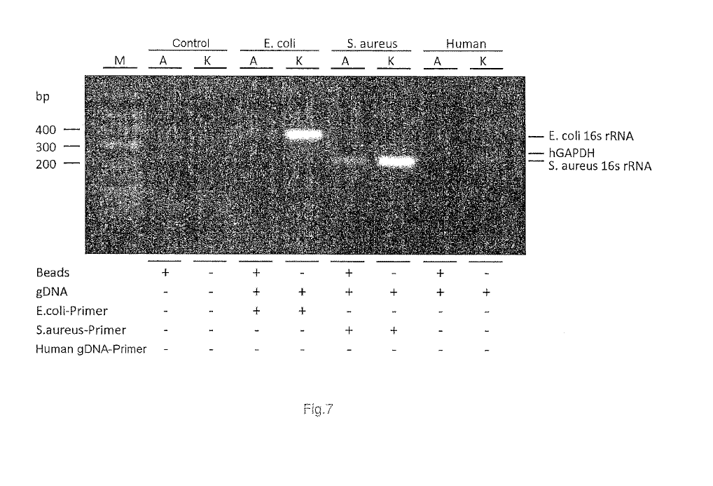

FIG. 8 shows a graphical plot of the efficiency of the isolation of DNA from Staphylococcus aureus from spiked buffer solutions using gyrA beads (magnetic particles: MagPrep®-P25 carboxy, φ25 nm, Merck, Darmstadt).

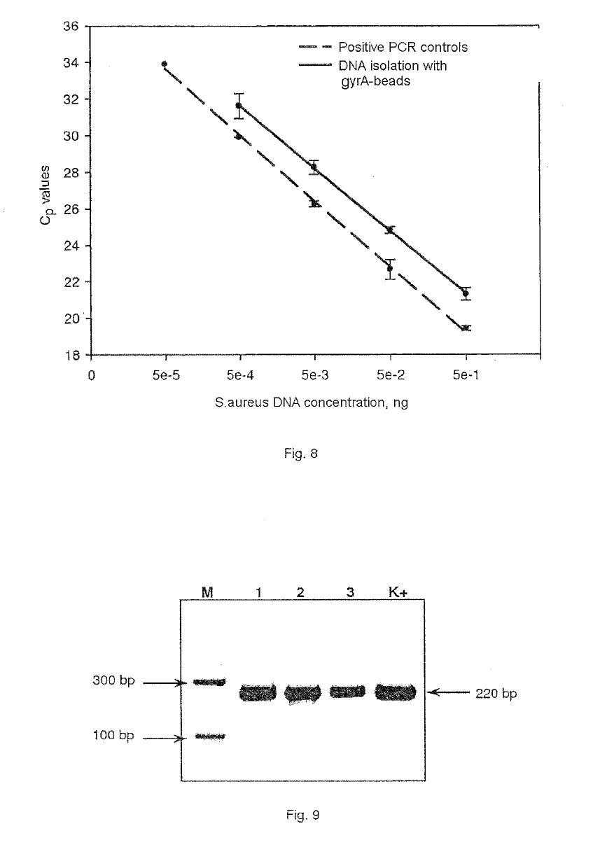

FIG. 9 shows isolation and detection of increasing concentrations of DNA from Staphylococcus aureus. The test solutions 1 to 3 each contained 20 μg of human DNA and were spiked with: (1) 0.5 ng, (2) 0.05 ng and (3) 0.005 ng of S. aureus DNA. (M): DNA length marker; K+: positive PCR control (0.005 ng of S. aureus DNA).

### REFERENCE

Mizuuchi, K., Mizuuchi, M., O'Dea, M. H. and Gellert, M. (1984). Cloning and simplified purification of Escherichia coli DNA gyrase A and B proteins. J. Biol, Chem. 259 (14): 9199-201.

1. An affinity material, comprising a support and a protein bound thereto, characterized in that the protein comprises the DNA-binding domain of a prokaryotic topoisomerase.

2. An affinity material according to claim 1, characterized in that the protein comprises the subunit A of the prokaryotic topoisomerase II.

3. An affinity material according to claim 1, characterized in that the support is developed in the form of magnetic particles, membranes, Sepharose and/or dextran.

4. An affinity material according to claim 1, characterized in that the protein is bound to the support by covalent bonding and/or by noncovalent interaction, especially in the form of protein-protein interactions and/or protein-polysaccharide interactions.

5. An affinity material according to claim 1, characterized in that the protein is a fusion protein which comprises the DNA-binding domain of a prokaryotic topoisomerase and an affinity tag.

6. An affinity material according to claim 1, characterized in that the protein comprises a fusion protein of the subunit A of the prokaryotic topoisomerase II and a maltose-binding protein.

7. An affinity material according to claim 1, characterized in that the protein has a sequence homology of ≧60%, especially ≧80%, especially ≧90% with the amino acid sequence according to SEQ-ID No. 1, especially preferably in that the protein has the amino acid sequence according to SEQ-ID No. 1.

8. A kit for enriching and/or separating prokaryotic DNA, comprising an affinity material according to claim 1.

9. A kit according to claim 8, characterized in that the kit comprises means for amplifying the enriched/separated prokaryotic DNA, especially in the form of primers for carrying out a polymerase chain reaction.

10. A method for separating off and/or enriching prokaryotic DNA, comprising the steps of:
a) contacting at least one prokaryotic DNA situated in solution with a binding agent that comprises the DNA-binding subunit of a prokaryotic topoisomerase,
b) separating the prokaryotic DNA that is bound to the binding agent.

11. A method according to claim 10, characterized in that the binding agent is present in the form of an affinity material comprising a support and a protein bound thereto that comprises the DNA-binding subunit of a prokaryotic topoisomerase.

12. A method according to claim 10, characterized in that the protein comprises the subunit A of the prokaryotic topoisomerase II.

13. A method according to claim 10, characterized in that magnetic particles, membranes, Sepharose and/or dextran are used as support.

14. A method according to claim 10, characterized in that the protein is bound to the support by covalent bonding and/or by noncovalent interaction, especially in the form of protein-protein interactions and/or protein-polysaccharide interactions.

15. A method according to claim 10, characterized in that the protein is a fusion protein which comprises a DNA-binding subunit of a prokaryotic topoisomerase and an affinity tag.

16. A method according to claim 10, characterized in that the protein comprises a fusion protein of the subunit A of the prokaryotic topoisomerase II and a maltose-binding protein.

17. A method according to claim 10, characterized in that a body fluid and/or a fluid derived from a body fluid is used as sample fluid, especially in the form of blood, serum, plasma, cell preparations from blood and/or sputum.

18. A method according to claim 10, characterized in that a fluid is used as sample fluid which is a food sample or environmental sample and/or a sample derived therefrom.

19. A method according to claim 10, characterized in that, after the step of separation of the prokaryotic DNA that is bound to the binding material, a step c is performed in which the prokaryotic DNA is subjected to an amplification, especially by polymerase chain reaction.

20. A method according to claim 1, characterized in that the protein has a sequence homology of ≧60%, especially ≧80%, especially ≧90%, with the amino acid sequence according to SEQ-ID No. 1, particularly especially in that the protein has the amino acid sequence according to SEQ-ID No. 1.

21. (canceled)

22. (canceled)
